(19) United States
(12) Patent Application Publication
(10) Pub. No.: US 2026/0284276 A1
(43) Pub. Date: Sep. 24, 2026

(54) IMPLANTABLE MEDICAL DEVICE INCLUDING POLYETHERURETHANE WITH IMPROVED BIOSTABILITY

(71) Applicant: MEDTRONIC, INC., Minneapolis, MN (US)

(72) Inventors: Julie A. ALKATOUT, Minneapolis, MN (US); Michael N. MISSAGHI, Edina, MN (US); Kimberly A. CHAFFIN, Woodbury, MN (US); Matthew JOLLY, Minneapolis, MN (US)

(21) Appl. No.: 19/168,890

(22) PCT Filed: Mar. 27, 2024
(86) PCT No.: PCT/US2024/021676

§ 371 Date: Sep. 25, 2025

Related U.S. Application Data
(60) Provisional application No. 63/455,301, filed on Mar. 29, 2023.

(51) Int. Cl. A61L 29/08; A61L 29/06; A61L 31/04; A61L 31/10

(52) U.S. Cl. CPC A61L 29/085; A61L 29/06; A61L 31/04; A61L 31/10; A61L 2420/02

(57) ABSTRACT

An implantable medical device includes a contacting substrate for contacting a bodily fluid or tissue. The contacting substrate includes polyetherurethane having a polyetherurethane surface. A layer of polysiloxane is chemically bonded to the polyetherurethane surface.

### TECHNICAL FIELD

This disclosure generally relates to an implantable medical device including polyetherurethane with improved biostability.

### BACKGROUND

There is a need for soft flexible polymers and constructs thereof with improved biostability for implantable medical devices, such as electrical leads, catheters, drug infusion catheters, etc. Widely adopted soft 80A durometer polyetherurethanes (for example, those available under the tradenames PELLETHANE, ELASTHANE, TECOTHANE) have desirable hydrolytic stability, mechanical properties, and processing characteristics which have led to their adoption in the implantable medical device industry. However, these materials are susceptible to oxidation, which is generally thought to occur from oxygen-based free radicals, originating from macrophages or foreign body giant cells when contacting a bodily fluid or tissue or catalyzed by metal ions. These radicals can extract a hydrogen atom from the methylene group adjacent to the ether functionality in the soft segment of the polyetherurethane, resulting in either chain scission or crosslinking. The oxidative processes observed in vivo with polyurethanes or polyetherurethanes are commonly referred to as Environment Stress Cracking (ESC) and Metal Ion Oxidation (MIO).

Over-coating metal lead conductors with fluoropolymers have successfully been employed to mitigate MIO. However, there may be circumstances where the medical device may need to be fluoropolymer-free. In addition, with ESC, cell-mediated oxygen-based free radicals are reactive and consequently react very quickly at the surface prior to diffusion and reaction in the bulk when contacting bodily fluids or tissue. This results in a heterogeneous oxidative attack that takes place primarily at the surface of the polyetherurethane giving rise to the formation of surface crazing and micro-cracks. Surface cracks can expose the underlying material to the oxidizing environment leading to an apparent acceleration of this process.

Since the observations back in the early 1980s of oxidative degradation of soft polyetherurethanes, the implantable medical device industry has continuously explored soft 80A durometer polyurethanes with improved oxidative stability, such as poly(dimethyl siloxane)-polyurethanes (for example, those available under the trade names ELAST-EON and PURSIL) and polycarbonate-polyurethanes (for example, that available under the tradename BIONATE or CARBOTHANE). However, despite their improved oxidative stability, for various reasons, these materials have not been widely adopted.

Polycarbonate-polyurethane showed superior oxidation stability compared to polyether-polyurethane; however, they are known to be susceptible to hydrolysis of the carbonate group in the soft segment. PDMS-polyurethanes have been proven to be susceptible to hydrolysis resulting in decreasing mechanical strength. It should be noted that hydrolysis, unlike oxidation, typically occurs throughout the polymer as water can readily diffuse into the bulk and consequently affects bulk mechanical properties, whereas cell-mediated oxidation takes place primarily at the surface. Further, the relative timescales of hydrolysis and oxidation in vivo can be different. For example, the cell-mediated inflammatory response that results in oxidation is typically most acute within the first few weeks and months of implantation, which recruits the cellular response that is the source of the oxidative free radicals. After this initial period, the implanted device typically becomes encapsulated by host tissue growth (fibrotic capsule), reducing the inflammation and related oxidation action. Whereas hydrolysis would be expected to occur more uniformly throughout the implant life, because water is pervasive and quickly reaches equilibrium concentrations within the polymer, hydrolysis remains relatively constant over time.

There is a need for safe materials that have the desirable hydrolytic stability and mechanical properties of soft polyetherurethanes while improving their oxidative resistance and mechanical stability.

### SUMMARY

This disclosure generally relates to an implantable medical device including polyetherurethane with improved biostability. This disclosure relates to an implantable medical device including polyetherurethane with a siloxane layer coating the surface of the polyetherurethane. The siloxane layer is chemically bonded to a surface of the polyetherurethane. The siloxane layer improves the biostability of the polyetherurethane without changing the desirable mechanical properties of the polyetherurethane.

The siloxane layer may be plasma deposited onto the polyetherurethane. The siloxane layer may be plasma polymerized and deposited onto the polyetherurethane. The siloxane layer may be in direct contact with and chemically bonded to the polyetherurethane surface. The siloxane layer may have a thickness less than 1 micrometer. The polyetherurethane coated with the siloxane layer may be extensible and resilient.

In one example, an implantable medical device includes a contacting substrate for contacting a bodily fluid or tissue, the contacting substrate including polyetherurethane having a surface. A layer of polysiloxane is chemically bonded to the surface of the polyetherurethane.

In another example, a method of manufacturing an implantable medical device includes plasma depositing a layer of polysiloxane onto a polyetherurethane surface. The layer of polysiloxane is covalently bonded to the polyetherurethane surface forming a coated surface. Then the method includes assembling the coated surface into an implantable medical device.

In another example, an implantable medical lead includes an elongated lead body having an outer surface for contacting a bodily fluid or tissue. The outer surface includes polyetherurethane having a polyetherurethane surface. A layer of polysiloxane is covalently bonded to the polyetherurethane surface. The layer of polysiloxane has a thickness of less than 1000 nanometers. An electrical conductor is disposed within the elongated lead body.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a schematic diagram of an illustrative cardiac therapy system.

FIG. 1B is a schematic diagram of another illustrative neurostimulator system.

FIG. 2A is a schematic diagram of another illustrative drug delivery system.

FIG. 2B is a schematic diagram of another illustrative drug delivery system.

FIG. 3A is a schematic perspective view of an illustrative elongated lead body.

FIG. 3B is a schematic cross-sectional view of the illustrative elongated lead body of FIG. 3A.

FIG. 4A is a schematic perspective view of an illustrative catheter body.

FIG. 4B is a schematic cross-sectional view of the illustrative catheter body of FIG. 5 is a graph of the molecular weight change measured as M₀/Mₙ (GPC) versus the thickness of the siloxane coating of Examples 5 to 8.

### DETAILED DESCRIPTION

This disclosure generally relates to an implantable medical device including polyetherurethane with improved biostability. This disclosure relates to an implantable medical device including soft polyetherurethane with a siloxane layer coating the surface of the polyetherurethane.

Soft polyetherurethanes (having a Shore A hardness in a range from 50 to 95) are desirable for use in implantable medical devices. However, these soft polyetherurethanes become susceptible to oxidation. Oxidation of the polyetherurethane leads to cracking and degradation of the mechanical properties of the polyetherurethane.

The siloxane coating layer described herein improves the biostability of the underlying polyetherurethane without changing the desirable mechanical properties of the polyetherurethane. Polysiloxane coated polyetherurethane is surprisingly extensible and biostable.

The term “implantable medical device” refers to a device that has surfaces that contact bodily fluid or tissue in the course of their operation, which are implanted into a body, and remain in the body for an extended period (for example, greater than 28 days). Examples include pacemakers, cardiac defibrillators, neuromodulation stimulators, drug infusion catheters, and the associated lead bodies and catheter bodies thereof.

All headings provided herein are for the convenience of the reader and should not be used to limit the meaning of any text that follows the heading unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (for example, 1 to 5 includes 1, 1.5, 2, 2.75, 3, 3.80, 4, 5, etc.)

As used herein, the terms “polyetherurethane” or “PEU” or “polyether-polyurethane” are interchangeable. Soft 80A durometer polyetherurethane (for example, those available under the trade designations PELLETHANE, ELASTHANE, TECOTHANE) have desirable hydrolytic stability, mechanical properties, and processing characteristics which have led to their adoption in the implantable medical device industry. However, these materials are susceptible to oxidation, which is generally thought to occur from oxygen-based free radicals, originating from macrophages or foreign body giant cells or catalyzed by metal ions. These radicals can extract a hydrogen atom from the methylene group adjacent to the ether functionality in the soft segment, resulting in either chain scission or crosslinking. The oxidative processes observed in vivo with polyurethanes are commonly referred to as Environment Stress Cracking (ESC) and Metal Ion Oxidation (MIO).

Polyetherurethane materials have been used in the production of implantable medical devices. For example, polyetherurethane may be made from methylene diphenyl diisocyanate (MDI), butane diol (BDO), and polyether macrodiols such as poly(tetramethylene oxide) (PTMO). Examples of such polyetherurethane include those available under the trade designations ELASTHANE 80A & 55D from DSM Biomedical, and PELLETHANE 236380A & 236355D from Lubrizol. ELASTHANE 80A/PELLETHANE 80A polyetherurethane possesses about 60% by weight of polyether content. Typically, the polyether forms the soft segment of the polymer (that is, a portion of the polyetherurethane and has a glass transition temperature (T_{g}) that is below body temperature). The polyetherurethanes described herein may have a Shore A hardness of about 50 to 95, or from about 70 to 90, or from 75 to 85.

The polyetherurethane may have a polyether content of about 40% or greater. The polyetherurethane may have a polyether content of about 50% or greater. The polyetherurethane may have a polyether content in a range from about 40% to about 80%. The polyetherurethane may have a polyether content in a range from about 50% to about 70%. The polyetherurethane may have a polyether content in a range from about 55% to about 65%.

In one example, an implantable medical device includes a contacting substrate for contacting a bodily fluid. The contacting substrate includes polyetherurethane having a polyetherurethane surface. A layer of polysiloxane is chemically bonded to the polyetherurethane surface.

A “contacting substrate” is an element of an implantable medical device that has a surface contacting bodily fluid or tissue in the course of its operation. Useful contacting substrates define an outer surface of a lead body, catheter body, and the like. The contacting substrates may define a cylindrical outer surface extending along a longitudinal axis of a lead body, catheter body, and the like.

The polysiloxane layer chemically bonded to the polyetherurethane surprisingly retains the mechanical properties of the polyetherurethane alone while improving the oxidative resistance of the polyetherurethane. The polysiloxane layer chemically bonded to the polyetherurethane significantly prevents or mitigates the propagation of cracks in the polyetherurethane as compared to uncoated polyetherurethane.

The polysiloxane layer may be formed by plasma deposition. This may be accomplished by plasma depositing a layer of polysiloxane onto a polyetherurethane surface. The layer of polysiloxane may be covalently bonded to the polyetherurethane surface forming a coated surface. This coated surface is then assembled into an implantable medical device.

Plasma deposition of polysiloxane advantageously forms a thin layer of polysiloxane on the polyetherurethane surface. This layer is very thin compared to the overall thickness of the polyetherurethane contacting substrate. Since the polysiloxane layer forms a minor portion of the overall thickness of the polyetherurethane contacting substrate, the overall mechanical properties of the polyetherurethane contacting substrate are maintained while increasing the biostability of the polyetherurethane contacting substrate. For example, the polyetherurethane contacting substrate may have a thickness of at least 100 times greater, at least 250 times greater, or at least 500 times greater, than the thickness of the polysiloxane layer.

In some examples, the polyetherurethane surface is cleaned to improve the deposition of the layer of polysiloxane. The surface of the polyetherurethane may be plasma cleaned prior to depositing the layer of polysiloxane on the polyetherurethane surface.

Plasma deposition may polymerize the siloxane monomers from the polysiloxane onto the polyetherurethane surface. Plasma polymerization uses plasma sources to generate a gas discharge that provides energy to activate or fragment gaseous or liquid monomer, to initiate polymerization. Plasma polymerization directly deposits a very thin, dense film on substrates. The plasma is formed by ionization of an organic monomer gas through an electrical discharge at high frequencies. Plasma polymerization takes place in three stages: (i) the initiation stage, where atoms and free radicals are generated due to the collision of electrons and ions with the gas monomers; (ii) the propagation stage, which is the formation of the polymeric chain; and (iii) the termination process, which is necessary to close the polymer chain.

Polymers formed from this plasma deposition technique may be highly branched and highly cross-linked and adhere to solid surfaces well. One advantage to this process is that polymers can be directly attached to a desired surface while the chains are growing, which reduces the steps necessary for other coating processes such as grafting. Plasma polymerization forms uniform pin-hole-free coatings having a thickness of less than 1 micrometer.

In many embodiments, the polysiloxane layer is formed by plasma polymerization of a siloxane-based monomeric gas such as hexamethyldisiloxane, trimethylsilane, hexamethyldisilazane, trimethylsilane, and the like.

In some embodiments, the siloxane-based monomeric gas or siloxane monomer includes at least one monomer including hexamethyldisiloxane. In some embodiments, hexamethyldisiloxane is the only monomer utilized to form the polysiloxane layer.

The plasma deposition may include comonomers with the hexamethyldisiloxane monomer. Comonomers may have a vapor pressure of greater than 3 torr at room temperature, be compatible with hexamethyldisiloxane as a comonomer, be reactive with polyetherurethane, be nontoxic, and be compatible with plasma reactor materials and components.

Useful comonomers include one or more silanes or organometallics such as trimethylsilane; hexamethyldisilazane; trimethylsilane and allyl amine mixture; or 1,1,3,3-tetramethyldisiloxane. Organic comonomers may include one or more of tetrafluoromethane; methane; acetylene; allyl amine; allyl alcohol; propyl amine; acrylic acid; nitrogen; water; propylene; hydrogen peroxide; or ethylene oxide.

The polyetherurethane contacting substrate may define or form an elongated lead body. The elongated lead body may contain an electrical conductor.

The polyetherurethane contacting substrate may have an outer polyetherurethane surface and an inner polyetherurethane surface. An inner polysiloxane layer may be chemically bonded to the inner polyetherurethane surface. An outer polysiloxane layer may be chemically bonded to the outer polyetherurethane surface.

The outer polysiloxane layer may form a uniform coating about the outer polyetherurethane surface. The inner polysiloxane layer may cover the entire inner polyetherurethane surface.

The outer polysiloxane layer may have a thickness of less than 1000 nanometers. The outer polysiloxane layer may have a thickness of less than 900 nanometers, or less than 750 nanometers, or less than 500 nanometers. The outer polysiloxane layer may have a thickness in a range from 100 nanometers to 900 nanometers, or from 100 nanometers to 500 nanometers, or from 200 nanometers to 750 nanometers, or 200 nanometers to 500 nanometers, or from 200 nanometers to 400 nanometers, or from 200 nanometers to 300 nanometers.

The inner polysiloxane layer may have a thickness of less than 1000 nanometers. The inner polysiloxane layer may have a thickness of less than 900 nanometers, or less than 750 nanometers, or less than 500 nanometers. The inner polysiloxane layer may have a thickness in a range from 100 nanometers to 900 nanometers, or from 100 nanometers to 500 nanometers, or from 200 nanometers to 750 nanometers, or 200 nanometers to 500 nanometers, or from 200 nanometers to 400 nanometers, or from 200 nanometers to 300 nanometers.

In some embodiments, the electrical conductor has a separate electrically insulating layer surrounding the electrical conductor and separating the electrical conductor from the elongated lead body. The polysiloxane layer may separate substantially an entire length of the electrical conductor from the elongated lead body.

In other embodiments, the electrical conductor is a “bare” electrical conductor that directly contacts the elongated lead body. In these embodiments, a polysiloxane layer may separate the electrical conductor from the elongated lead body. The polysiloxane layer may separate substantially an entire length of the electrical conductor from the elongated lead body.

The polyetherurethane lead body may have a thickness range from 50 micrometers to 5000 micrometers, or from 100 micrometers to 1000 micrometers.

The polyetherurethane and layer of polysiloxane of the elongated lead body are extensible with an elastic elongation of at least 10%. The polyetherurethane and layer of polysiloxane of the elongated lead body are extensible with an elastic elongation of at least 15%. The polyetherurethane and layer of polysiloxane of the elongated lead body are extensible with an elastic elongation of at least 20%.

When the contacting substrate is an elongated lead body, the implantable medical device may be a neurostimulation lead, a cardiac pacing lead, a defibrillator lead, and the like.

The polyetherurethane contacting substrate may form a tubular member. The tubular member may define an elongated cylinder extending along a longitudinal axis. The tubular member may be defined by an inner polyetherurethane cylindrical surface and an outer polyetherurethane cylindrical surface. The inner polyetherurethane cylindrical surface defines a cylindrical void space. The cylindrical void space may be co-extensive with the length of the tubular member.

The polyetherurethane may form the entire tubular member. The polyetherurethane may form the entire thickness of the tubular member. The tubular member may have a uniform outer diameter. The tubular member may have a uniform inner diameter.

An outer polysiloxane layer may be chemically bonded to the outer polyetherurethane cylindrical surface. The outer polysiloxane layer may form a uniform coating about the outer polyetherurethane cylindrical surface. The outer polysiloxane layer may cover the entire outer polyetherurethane cylindrical surface.

The outer polysiloxane layer may have a thickness of less than 1000 nanometers. The outer polysiloxane layer may have a thickness of less than 900 nanometers, or less than 750 nanometers, or less than 500 nanometers. The outer polysiloxane layer may have a thickness in a range from 100 nanometers to 900 nanometers, or from 100 nanometers to 500 nanometers, or from 200 nanometers to 750 nanometers, or 200 nanometers to 500 nanometers, or from 200 nanometers to 400 nanometers, or from 200 nanometers to 300 nanometers.

An inner polysiloxane layer may be chemically bonded to the inner polyetherurethane cylindrical surface. The inner polysiloxane layer may form a uniform coating about the inner polyetherurethane cylindrical surface. The inner polysiloxane layer may cover the entire inner polyetherurethane cylindrical surface.

The inner polysiloxane layer may have a thickness of less than 1000 nanometers. The inner polysiloxane layer may have a thickness of less than 900 nanometers, or less than 750 nanometers, or less than 500 nanometers. The inner polysiloxane layer may have a thickness in a range from 100 nanometers to 900 nanometers, or from 100 nanometers to 500 nanometers, or from 200 nanometers to 750 nanometers, or 200 nanometers to 500 nanometers, or from 200 nanometers to 400 nanometers, or from 200 nanometers to 300 nanometers.

The layer of polysiloxane coating the inner polyetherurethane surface of a tubular member and the layer of polysiloxane coating the outer polyetherurethane surface of a tubular member may be plasma deposited at the same time.

The polyetherurethane tubular member may have a thickness in a range from 50 micrometers to 5000 micrometers, or from 100 micrometers to 1000 micrometers.

The polyetherurethane and layer of polysiloxane of the tubular member are extensible with an elastic elongation of at least 10%. The polyetherurethane and layer of polysiloxane of the tubular member are extensible with an elastic elongation of at least 15%. The polyetherurethane and layer of polysiloxane of the tubular member are extensible with an elastic elongation of at least 20%.

The polysiloxane coated polyetherurethane tubular member may form an element of a catheter or a drug delivery catheter. The polysiloxane coated polyetherurethane tubular member may form an elongated and flexible body portion of the catheter.

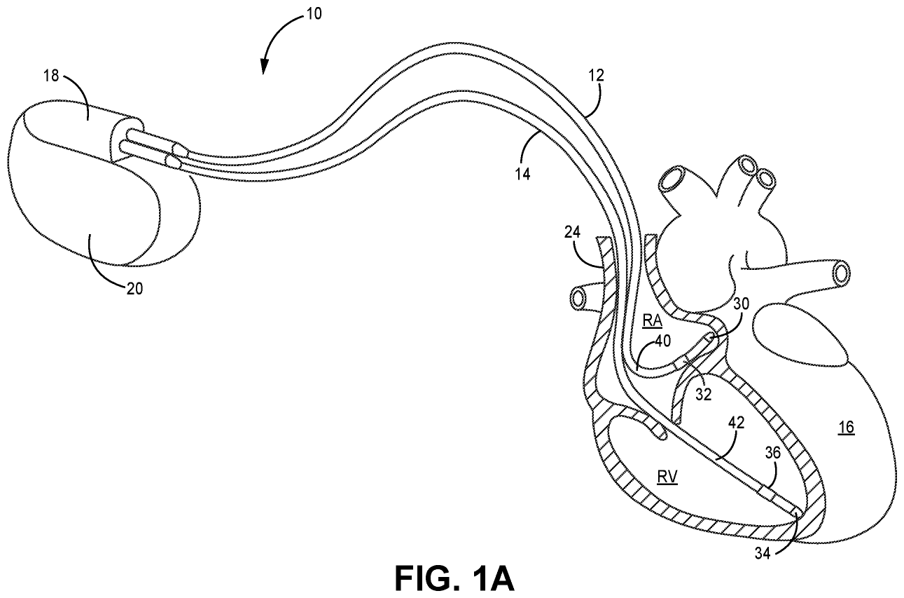

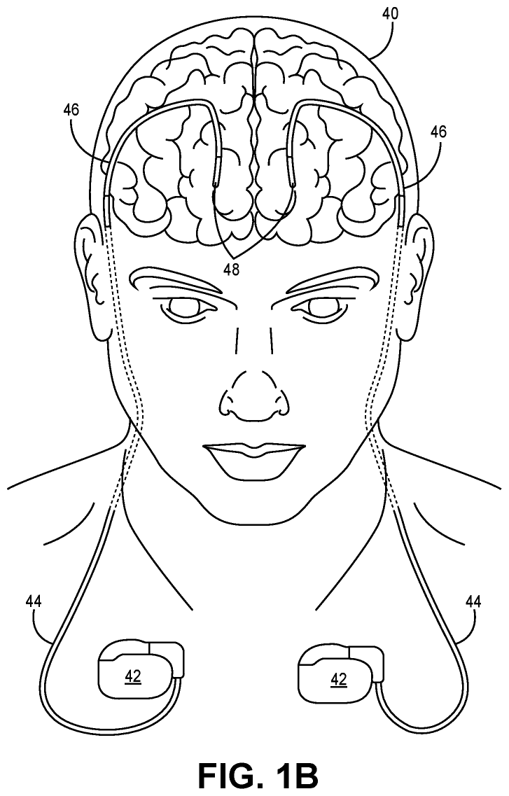

FIG. 1A is a schematic view of an exemplary cardiac therapy system 10. The cardiac therapy system 10 includes an atrial lead 12 and ventricular lead 14 implanted in heart 16 (for example, as described in greater detail in U.S. Pat. No. 7,780,607). The cardiac therapy system 10 may be a pacemaker, defibrillator, cardioverter, pacemaker/cardioverter/defibrillator (PCD), heart function monitor having pacing capabilities, or other implantable devices that include the capability of providing therapy to the heart 16. The cardiac therapy system 10 includes a connector module or header 18 and housing 20. Atrial lead 12 and ventricular lead 14 extend from connector module 18 into the right atrium RA and right ventricle RV, respectively, of heart 16. Proximal ends of atrial lead 12 and ventricular lead 14 are connected at header 18 to sensing, signal processing, and therapy delivery circuitry (not shown) within housing 20. Atrial lead 12 and ventricular lead 14 enter the right atrium RA through the superior vena cava 24. Atrial lead 12 is a J-shaped bipolar lead including tip electrode 30 and ring electrode 32 at its distal end, while ventricular lead 14 is an elongated bipolar lead including tip electrode 34 and ring electrode 36 at its distal end. While bipolar leads 12 and 14 are disclosed, unipolar leads can alternatively be implanted in the same anatomic relation to the heart chambers.

When heart 16 contracts, atrial lead 12 and ventricular lead 14 are deflected. The atrial contraction causes bending or deformation of atrial lead 12 along bending portion 40, while the ventricular contraction causes bending or deformation of ventricular lead 14 along bending portion 42. The magnitude of the deflection along bending portions 40 and 42 depends on the radial stiffness of atrial lead 12 and ventricular lead 14, respectively, and the muscle contraction forces of heart 16. In addition, the magnitude of the deflection depends on the initial bending forces caused by the specific implantation position. For instance, atrial lead 12 implanted on the anterior atrial wall (as shown in FIG. 1A) has a larger J-shape radius than a lead implanted in the atrial appendage. Atrial lead 12 and ventricular lead 14 are strongly mechanically coupled to the heart muscle, especially in the chronic phase of cardiac pacing when fibrotic tissue anchors the lead tips to the endocardium.

The atrial lead 12 and ventricular lead 14 are lead bodies that contact bodily fluids or tissue. The atrial lead 12 and ventricular lead 14 may be formed of polysiloxane coated polyetherurethane as described herein.

FIG. 1B illustrates a neurostimulator system implanted in a patient 40 that includes at least one neurostimulator, at least one extension, and at least one stimulation lead containing electrodes (for example, as described in greater detail in EP1740260 B1). As can be seen, each neurostimulator 42 is implanted in the pectoral region of the patient. Extensions 44 are deployed up through the patient's neck and leads 46 are implanted in the patient's brain as shown at 48. As can be seen, each of the leads 46 is connected to its respective extension 44 just above the ear on both sides of patient 40.

The leads 46 may be formed of polysiloxane coated polyetherurethane lead as described herein.

Polysiloxane coated polyetherurethane tubular members may be used as catheter tubing, such as drug infusion catheter tubing (that is, drug delivery catheter tubing), hemodialysis catheter tubing, peritoneal catheter tubing, etc.

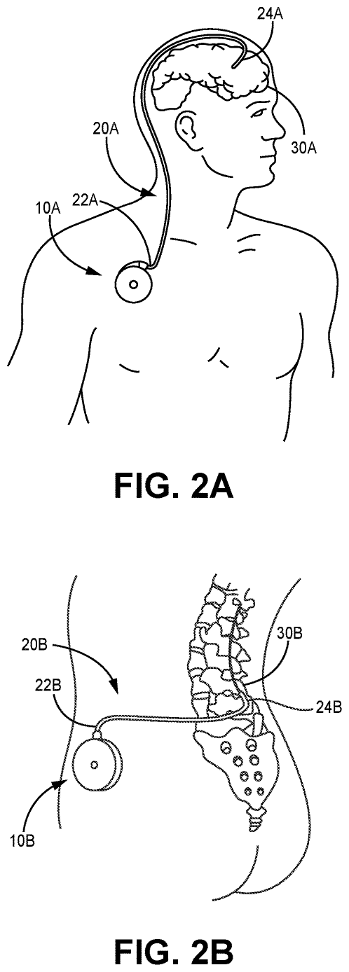

Exemplary embodiments of some drug delivery systems for infusing drugs are depicted in FIGS. 2A and 2B (for example, as described in greater detail in U.S. Pat. No. 8,043,281). FIG. 2A is a schematic diagram of a drug delivery system for infusing drugs into the brain, and FIG. 2B is a schematic diagram of a drug delivery system for infusing drugs into the spinal region.

The drug delivery systems depicted in FIGS. 2A and 2B include a drug infusion pump assembly 10A/10B and catheter 20A/20B having a proximal end 22A/22B attached to the pump assembly and distal end 24A/24B implanted within the patient. The distal end 24A is implanted within the brain 30A of the patient, while the distal end 24B is implanted within the spinal column 30B of the patient.

The polysiloxane coated polyetherurethane substrate forms the tubular body (of catheter 20A/20B in FIGS. 2A and 2B) having an inner surface and an outer surface, and the polysiloxane may be directly disposed on the outer surface of the tubular body.

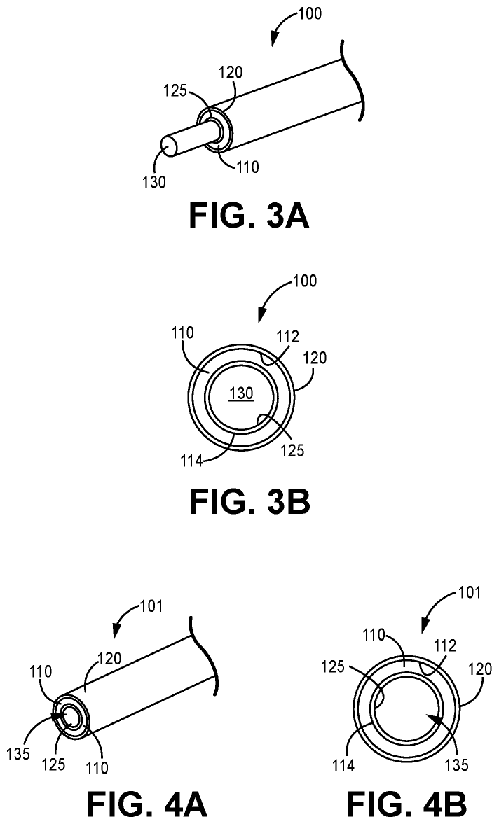

Coating both the outer and inner surfaces of the tubular body may be useful in preventing oxidative processes from occurring such as MIO (from exposed metal lead conductors) or long-term exposure to drug formulations (e.g., drug infusion catheters). FIG. 3B shows the polysiloxane coating both the inner (ID) and outer (OD) diameters.

FIG. 3A is a schematic perspective view of an illustrative elongated lead body 100. FIG. 3B is a schematic cross-sectional view of the illustrative elongated lead body 100 of FIG. 3A.

The elongated lead body 100 includes an electrical conductor 130 disposed in a contacting substrate 110. The contacting substrate 110 is disposed circumferentially about the electrical conductor 130 and is coextensive with the electrical conductor 130 along a length of the electrical conductor 130.

The contacting substrate 110 is formed of a soft polyetherurethane having a Shore A hardness of 50 to 95, or from 70 to 90. The soft polyetherurethane has a polyether content in a range from about 50% to about 70%.

The contacting substrate 110 has an outer polyetherurethane surface 112 and an inner polyetherurethane surface 114. An inner polysiloxane layer 125 is covalently bonded to the inner polyetherurethane surface 114. An outer polysiloxane layer 120 is covalently bonded to the outer polyetherurethane surface 112.

The inner and outer polysiloxane layers 120, 125 have a thickness in a range from 100 to 500 nanometers. The contacting substrate 110 has a thickness range from 100 to 1000 micrometers. The electrical conductor 130 is a bare conductive wire directly contacting the inner polysiloxane layer 125.

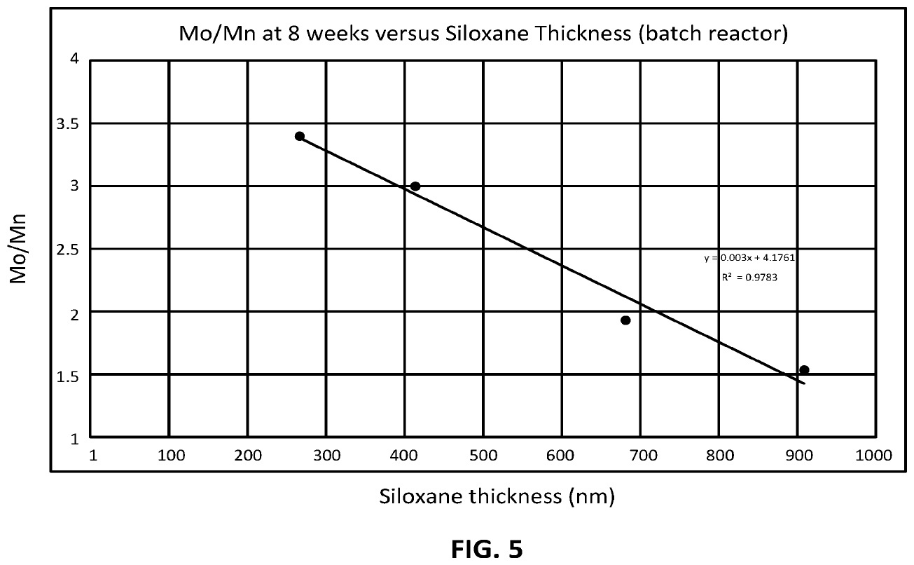

FIG. 4A is a schematic perspective view of an illustrative catheter body 101. FIG. 4B is a schematic cross-sectional view of the illustrative catheter body 101 of FIG. 4A.

The catheter body 101 defines a tubular member or contacting substrate 110. The tubular member or contacting substrate 110 is defined by an inner polyetherurethane cylindrical surface 114 and an outer polyetherurethane cylindrical surface 112. The inner polyetherurethane cylindrical surface 114 defining a cylindrical void space 135.

The contacting substrate or contacting substrate 110 is formed of a soft polyetherurethane having a Shore A hardness of 50 to 95, or from 70 to 90. The soft polyetherurethane has a polyether content in a range from about 50% to about 70%.

An inner polysiloxane layer 125 is covalently bonded to the inner polyetherurethane surface 114. An outer polysiloxane layer 120 is covalently bonded to the outer polyetherurethane surface 112.

The inner and outer polysiloxane layers 120, 125 have a thickness in a range from 100 to 500 nanometers. The tubular member or contacting substrate 110 has a thickness range from 100 to 1000 micrometers. The cylindrical void space 135 is configured to flow liquid drugs to a drug delivery site.

### EXAMPLES

Objects and advantages of this disclosure are further illustrated by the following examples, but the particular materials and amounts thereof recited in these examples, as well as other conditions and details, should not be construed to unduly limit this disclosure.

### Materials

Thermoplastic polyetherurethane elastomer under the trade designation PELLETHANE 80A tubing was extruded by Medtronic (Danvers, MA) for the continuous and batch plasma reactor to an outer diameter of 1.27 mm and an inner diameter of 0.79 mm with a wall thickness of 0.24 mm.

Hexamethyldisiloxane (HMDSO), NMR grade, 99.7% (catalog no. L16970) was purchased from Alfa Aesar (Ward Hill, MA).

### Methods

Plasma Treatment (Batch Reactor Method)—Polyetherurethane material to be plasma coated (either tubing or flat “dog bone” stampings of a cast sheet) was fixtured using gold-plated copper clips and/or nitinol wire in an open rectangular frame, and inserted horizontally into a plasma cleaning chamber, with a biased RF electrode 1-2 inches above the sample, and a grounded RF electrode a similar distance below the sample. The polyetherurethane sample was evacuated to a base pressure of 20 mTorr, whereupon oxygen and/or argon gas was introduced at a ratio varying from pure oxygen to pure argon, and with a total flow rate of 10-50 standard cubic centimeters per minute (sccm). The flow pattern in the reactor was top to bottom. The pressure in the chamber was regulated using downstream throttle valve control to a steady-state pressure of between 100 and 200 mTorr. At this point, the 13.56 MHz RF plasma generator was switched on with a power setting of between 50 and 225 W, and the plasma cleaning process was conducted with a duration ranging from 10 seconds to 5 minutes. After the completion of the batch cleaning cycle on the polyetherurethane sample, the vacuum pump and flow controllers were switched off and the reactor was vented with nitrogen to atmospheric pressure.

The rectangular holding fixture and polyetherurethane sample were moved from the cleaning chamber to an adjacent vacuum chamber which was dedicated to plasma deposition. Again, the chamber was pumped down to a base pressure of 20 mTorr, and hexamethyldisiloxane, argon, and nitrous oxide flowed into the chamber, typically in a molar ratio of 10:1:30, respectively. The pressure in the chamber was regulated using downstream throttle valve control to a steady-state pressure of between 100 and 200 mTorr. At this point, the 13.56 MHz RF plasma generator was switched on with a typical power setting of 140 W, and the plasma deposition process was conducted with a typical duration of 100 seconds onto the polyetherurethane sample. In some experimental trials, multiple deposition cycles were performed to obtain a thicker coating on the polyetherurethane sample. Typically, this was done after venting the plasma chamber and allowing for the material to cool to ambient temperature, to prevent thermal damage to the polyetherurethane sample substrate.

Plasma Treatment (Continuous Reactor Method)—Polyetherurethane tubing material to be treated was level wound onto an anodized aluminum process spool and loaded into a continuous plasma reactor. The reactor consisted of an upper payoff chamber, a lower takeup chamber, and a series of vertical glass tubes and metal transition fittings in between, with appropriate connections for gas inlet flow and vacuum exhaust. The polyetherurethane tubing material was threaded through the middle part of the reactor and gripped by a pinch roller mechanism which served to pull the tubing through the reactor and deposit it in a metal collection basket in the bottom chamber. The system was evacuated to a base pressure of 10 mTorr, after which gas flow into the various reactor zones was turned on. A mixture of argon and oxygen gas in a typical molar ratio of 3:1 and with a typical combined flow rate of 8 sccm flowed into an upper plasma cleaning zone consisting of a 1.5-inch OD borosilicate glass tube with compression metal fittings, outside of which was placed an inductive plasma electrode coil with an outer ground sheath. Simultaneously, a mixture of hexamethyldisiloxane, argon, and nitrous oxide with a typical molar ratio of 10:1:30 and a typical combined flow rate of 12 sccm was introduced into a lower plasma deposition zone, consisting of a 1.5-inch OD borosilicate glass tube with compression metal fittings, outside of which was placed a capacitive three-ring electrode with a single RF-biased ring in the center and grounded rings at either end. The direction of gas flow in both zones was top-to-bottom (the same as the direction of tubing transport). Typically, a zone pressure of 0.10-0.15 Torr was used for these studies. The polyetherurethane tubing transport speed ranged from 10-45 inches per minute, and the 13.56 MHz RF power settings for the cleaning and deposition zones ranged from 10-75 W. Care was taken to avoid excessive heating of the polyetherurethane tubing from exposure to the plasma emission from the two reactor zones, which could result in melting or surface texture changes in the material. After the desired amount of material had been transported through the plasma cleaning and deposition zones, the RF generators, flow controllers, motors, and vacuum pump were switched off, and the system was vented with nitrogen to atmospheric pressure.

Plasma Polymer Film Thickness Measurement—The polymer sample to be measured was mounted on a 32 mm aluminum scanning electron microscope (SEM) sample holder (Ted Pella, part no. 16148) with the aid of a conductive adhesive pad (Ted Pella, part no. 16084-2). Conductive silver paste (Ted Pella, part no. 16062-15) was dabbed liberally onto one end of the sample to promote grounding. The sample holder was placed in a sputter coater (Denton Vacuum Desk II) and coated with an ultra-thin gold-palladium film. The sample was then moved to a focused ion beam scanning electron microscope (FIB-SEM, FEI). After evacuating the sample chamber, a suitable region of the sample was found for analysis, and a bar of platinum was deposited using a volatile platinum deposition source, with a deposition width of 10 micrometers, and deposition length of 2 micrometers, and a target deposition thickness of ca. 1 micrometer. Next, a wedge-shaped trench was milled out of the sample, with the shallow end distal from the platinum bar, and the deep end intruding a slight amount into the platinum bar, with a target maximum depth of 2-3 micrometers. Then a second trench was milled with a stepper pitch and opposite orientation, starting on the platinum bar side and extending into the exposed sample. After these FIB operations, a cross-section of material was exposed in which the thin film was sandwiched between the platinum layer and the substrate. The thin film was measured using the calibrated distance measurement feature of the SEM with automatic correction for the tilt angle of the cross-section in the field of view.

The Oxidation Test—This is done using an in vitro accelerated oxidation test commonly known as the “Stokes Test” developed by Ken Stokes and Jim Anderson (Schubert et al., J. Biomed. Mater. Res., 34, 519-530 (1997), and Zhao et al., J. Biomed. Mate.r Res., 29, 467-475 (1995)). It is recognized that there are several variants of this test, however, for comparison, the test used to compare materials of this invention (“the oxidation test”) is hydrogen peroxide (20 wt.-%)/(0.1 M cobalt chloride hexahydrate) solution at 37 degrees Celsius for up to 16 weeks (without glass wool). The solution was changed every 3-4 days. The samples are monitored periodically (typically, at 2, 4, and 8 weeks) and at the end of these 16 weeks for changes compared with control unexposed samples (surface morphology, bulk mechanical properties, chemical, etc.). The cobalt chloride catalyzes the formation of reactive oxygen-based free radicals from hydrogen peroxide (hydroxyl (OH), superoxide (O-2), peroxyl (HOO), etc.) which can readily attack the polyether soft segment of polyetherurethane. Many of these reactive oxygen-based free radicals are believed to be similar to those secreted by inflammatory cells (macrophages, foreign body giant cells, etc.) in vivo. The test has been shown to accelerate the effects of oxidation in vivo with similar changes observed chemically and in surface morphology (Christenson et al., J. Biomed. Mater. Res., 70A, 245-255 (2004)).

Uncoated polyetherurethane samples and polysiloxane coated polyetherurethane samples were immersed in hydrogen peroxide (20 wt.-%)/(0.1 M cobalt chloride hexahydrate) solution at 37 degrees Celsius and the solution changed every 3-4 days to maintain reactive species. At each time point a sample was removed from the solution, rinsed with DI water, and vacuum oven dried at 45° C. for 36 hours before being evaluated. Samples were evaluated after 8 and 16 weeks for appearance (optical and SEM imaging), polyurethane molecular weight (Molar Mass Measured by Gel Permeation Chromatography or GPC), mechanical properties, surface chemistry and wear abrasion.

Tensile Test—Tensile evaluation was performed via ASTM D638-10 using an MTS Sintech 1/D test frame with an extensometer. Samples were pulled at a crosshead speed of 5 inches/min (n=10). The samples were tested in a hydrated condition at ambient temperature and pressure.

Fatigue Test (Dry and Wet)—Adjust grip separation to 5 inches on the MTS fatigue system. Load 4 tubing samples, 7 or more inches each, in the MTS grips, ensuring all samples are firmly secure in the upper and lower grips. Program software to perform elongation at + and −10% for 150,000 cycles at one cycle per second. If performing hydrated or wet fatigue, ensure that the water bath (DI water) is stabilized at the desired temperature, close the water bath fixture surrounding the MTS grips, and open the flow valves to deliver the water to the fatigue samples. Start the test cycle once the water has filled the chamber.

Molecular Weight Change Test (Molar Mass or Measured by Gel Permeation Chromatography or GPC)—Samples were dissolved in tetrahydrofuran (THF) at a concentration of 5 mg/mL and 100 uL of the solution was injected into the GPC. All the samples were analyzed at one time to avoid the systematic deviation from instrument run-to-run variability. Each sample was injected twice and the reported numbers are average from the two runs.

The GPC system is comprised of an Agilent 1100 pump, three Phenogel columns (10E5A, 10E5A, 10E3A) connected in series, and a RI detector from Wyatt. The flow rate is set at 1 mL/min and the column temperature is set at 40° C. The GPC system is calibrated with 12 PS standards before injecting samples and a 30 kDa PS standard is injected every run as QC for the system.

Results are evaluated using M₀/Mₙ, where M₀ is the initial number averaged molecular weight (Mₙ), and Mₙ is the number averaged molecular weight at the time. MW is used as the abbreviation for molecular weight and should not be mixed with Mw (weight averaged molecular weight).

The initial molecular weight (number average molecular weight) of the polyetherurethane sample is recorded as M₀. The aged and/or fatigued molecular weight (number average molecular weight) of the polyetherurethane sample is recorded as Mₙ. An M₀/Mₙ value of “1” indicates no change in the molecular weight of the polyetherurethane sample due to aging and/or fatiguing. An M₀/Mₙ value of “2” indicates the molecular weight of the polyetherurethane sample has been cut in half due to aging and/or fatiguing (breaking down the initial polymer chains). An M₀/Mₙ value of “3” indicates the molecular weight of the polyetherurethane sample has been cut into thirds due to aging and/or fatiguing, and so forth. Higher values indicate greater polymer chain degradation.

Cyclical Abrasion Test Method—Samples that had experienced 8 weeks of accelerated oxidation testing as previously described and time zero controls were subjected to abrasion testing. Load-bearing cylindrical titanium coupons were used to simulate the rubbing contact between a lead and titanium components of medical devices. The geometry of the test samples was pressed sheet. The samples were moved vertically against the titanium coupon with a constant force. The abrasion testing was performed in a water bath at 37 Celsius. The abrasion tester has 4 stations to abrade 4 samples simultaneously, with 2 uncoated and 2 coated samples of the same time point tested in each trial. The size of the resulting wear scar was visually evaluated and measured to calculate the volume of material removed from the test samples.

### Example 1—Uncoated Polyetherurethane “Aged”

Uncoated PELLETHANE 80A polyetherurethane was “aged” with “the oxidation test” for 16 weeks and tested for appearance (optical and SEM imaging), and mechanical properties.

At 16 weeks the uncoated polyetherurethane tubing exhibited significant surface cracking when viewed with SEM imaging.

At 0 weeks the average tensile was about 55 MPa (stress) at 750% (strain) at break. At 16 weeks the average tensile was about 30 MPa (stress) at 800% (strain) at break.

### Example 2—Uncoated Polyetherurethane “Fatigued and Aged”

Uncoated PELLETHANE 80A polyetherurethane was either “dry fatigued” or “wet fatigued” using the Fatigue Test and then “aged” with the oxidation test. These samples were evaluated for appearance (optical and SEM imaging), and polyurethane molecular weight (GPC).

At 8 weeks the uncoated polyetherurethane tubing, both dry and wet fatigued samples, exhibited significant surface cracking when viewed with SEM imaging.

At 8 weeks the uncoated polyetherurethane dry fatigued sample had three times the molecular weight change (degradation) of the polysiloxane coated polyetherurethane dry fatigued sample: 4.5 M₀/Mₙvs. 1.5 M₀/Mₙ.

At 8 weeks the uncoated polyetherurethane wet fatigued sample had two times the molecular weight change (degradation) of the polysiloxane coated polyetherurethane wet fatigued sample: 11 M₀/Mₙ vs. 5 M₀/Mₙ.

### Example 3—Polysiloxane (250 nm) Coated Polyetherurethane “Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 250+/−50 nanometers.

Polysiloxane coated polyetherurethane was “aged” with “the oxidation test” for 8 weeks and tested for appearance (optical and SEM imaging), polyurethane molecular weight (GPC), mechanical properties, surface chemistry and wear abrasion.

At 8 weeks the polysiloxane coated polyetherurethane tubing exhibited reduced surface cracking compared to the untreated control when viewed with SEM imaging.

At 0 weeks the average tensile was about 50 MPa (stress) at 725% (strain) at break. At 8 weeks the average tensile was about 50 MPa (stress) at 900% (strain) at break.

The “aged” polysiloxane coated polyetherurethane exhibited superior mechanical properties as compared to the uncoated “aged” polyetherurethane.

### Example 4—Polysiloxane (250 nm) Coated Polyetherurethane “Fatigued and Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 250 +/−50 nanometers.

Polysiloxane coated polyetherurethane was either “dry fatigued” or “wet fatigued” using the Fatigue Test and then “aged” with the oxidation test. These samples were evaluated for appearance (optical and SEM imaging), and polyurethane molecular weight (GPC).

At 8 weeks the polysiloxane coated polyetherurethane tubing, both dry and wet fatigued, exhibited reduced surface cracking compared to the untreated control when viewed with SEM imaging.

At 8 weeks the polysiloxane coated polyetherurethane dry-fatigued sample exhibited a minimal decrease in molecular weight compared to the uncoated polyetherurethane dry-fatigued sample. The coated polyetherurethane dry fatigued sample has one-third the molecular weight change (degradation) of the uncoated polyetherurethane dry fatigued sample: 1.5 M₀/Mₙ vs. 4.5 M₀/Mₙ.

At 8 weeks the polysiloxane coated polyetherurethane wet fatigued sample exhibited a smaller decrease in molecular weight compared to the uncoated polyetherurethane wet fatigued sample. The coated polyetherurethane wet fatigued sample has one-half the molecular weight change (degradation) of the uncoated polyetherurethane wet fatigued sample: 5 M₀/Mₙ vs. 11 M₀/Mₙ.

Plasma polymerized siloxane on PELLETHANE 80A polyetherurethane tubing is surprisingly resilient to 10% elongation fatigue (both wet and dry) testing and better maintained mechanical properties while preventing oxidation of the soft segments of the polyetherurethane as compared to uncoated PELLETHANE 80A polyetherurethane tubing.

Cyclical Abrasion Results: Abrasion scars were smaller for the plasma-coated samples compared to the uncoated controls, at both time zero and after 8 weeks of accelerated oxidation testing. At time zero, the volume of the wear scar for the uncoated sample was 0.19 (stdev=0.11 ) and for the plasma-coated sample was 0.14 (stdev=0.05 ). For the 8-week oxidation samples, the uncoated sample had a wear scar volume of 0.155 mm³ (stdev=0.03) and the coated sample was 0.11 (stdev=0.05 ). The wear scars with siloxane coating present were 21% smaller with no oxidation exposure (time 0) and 30% smaller at 8 weeks of oxidation exposure. The mechanism of protection by plasma-polymerized siloxane may be increased lubricity at time zero and a combination of increased lubricity and protection of polyetherurethane from chemical degradation during the oxidation challenge.

### Example 5—Polysiloxane (910 nm) Coated Polyetherurethane “Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 909+/−50 nanometers.

Polysiloxane coated polyetherurethane was “aged” with “the oxidation test” for 8 weeks and tested for appearance (optical and SEM imaging) and polyurethane molecular weight (GPC).

At 8 weeks the polysiloxane coated polyetherurethane tubing exhibited no surface cracking when viewed with SEM imaging.

At 8 weeks the polysiloxane coated polyetherurethane sample exhibited a minimal decrease in molecular weight compared to the uncoated polyetherurethane sample: 1.5 M₀/Mₙ vs. 9.8 M₀/Mₙ.

### Example 6—Polysiloxane (680 nm) Coated Polyetherurethane “aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 681+/−50 nanometers.

Polysiloxane coated polyetherurethane was “aged” with “the oxidation test” for 8 weeks and tested for appearance (optical and SEM imaging) and polyurethane molecular weight (GPC).

At 8 weeks the polysiloxane coated polyetherurethane tubing exhibited minimal surface cracking when viewed with SEM imaging.

At 8 weeks the polysiloxane coated polyetherurethane sample exhibited a minimal decrease in molecular weight compared to the uncoated polyetherurethane sample: 1.9 M₀/Mₙ vs. 9.8 M₀/Mₙ.

### Example 7—Polysiloxane (410 nm) Coated Polyetherurethane “aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 413+/−50 nanometers.

Polysiloxane coated polyetherurethane was “aged” with “the oxidation test” for 8 weeks and tested for appearance (optical and SEM imaging) and polyurethane molecular weight (GPC).

At 8 weeks the polysiloxane coated polyetherurethane tubing exhibited greatly reduced surface cracking compared to the untreated control when viewed with SEM imaging.

At 8 weeks the polysiloxane coated polyetherurethane sample exhibited a minimal decrease in molecular weight compared to the uncoated polyetherurethane sample: 3.0 M₀/Mₙ vs. 9.8 M₀/Mₙ.

### Example 8—Polysiloxane (265 nm) Coated Polyetherurethane “Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 265+/−50 nanometers.

Polysiloxane coated polyetherurethane was “aged” with “the oxidation test” for 8 weeks and tested for appearance (optical and SEM imaging) and polyurethane molecular weight (GPC).

At 8 weeks the polysiloxane coated polyetherurethane tubing exhibited greatly reduced surface cracking compared to the untreated control when viewed with SEM imaging.

At 8 weeks the polysiloxane coated polyetherurethane sample exhibited a minimal decrease in molecular weight compared to the uncoated polyetherurethane sample: 3.4 M₀/Mₙ vs. 9.8 M₀/Mₙ.

### Example 9—Correlation between Polysiloxane Thickness and Molecular Weight Change of “Aged” Samples

The polyetherurethane tubing from Examples 5 to 8 was “aged” with “the oxidation test” for 8 weeks and tested for molecular weight change measured as M₀/Mₙ (GPC). The M₀/Mₙ was plotted versus the polysiloxane thickness (FIB/SEM) as illustrated in FIG. 5. The correlation was linear with an R² of 0.98 and the equation was y=−0.003x+4.1761. This demonstrates that the molecular weight change with “aging” is directly proportional to the thickness of polysiloxane on the polyetherurethane.

### Example 10—Polysiloxane (910 nm) Coated Polyetherurethane “Fatigued and Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 909+/−50 nanometers.

Polysiloxane coated polyetherurethane was “wet fatigued” using the Fatigue Test and then “aged” with the oxidation test. These samples were evaluated for appearance (optical and SEM imaging), and polyurethane molecular weight (GPC).

At 8 weeks the polysiloxane coated polyetherurethane tubing, wet fatigued, exhibited greatly reduced surface cracking compared to the untreated control when viewed with SEM imaging.

At 8 weeks the polysiloxane coated polyetherurethane wet fatigued sample exhibited a smaller decrease in molecular weight compared to the uncoated polyetherurethane wet fatigued sample: 3.8 M₀/Mₙ vs. 8.7 M₀/Mₙ.

### Example 11—Polysiloxane (680 nm) Coated Polyetherurethane “Fatigued and Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 909+/−50 nanometers.

Polysiloxane coated polyetherurethane was “wet fatigued” using the Fatigue Test and then “aged” with the oxidation test. These samples were evaluated for appearance (optical and SEM imaging), and polyurethane molecular weight (GPC).

At 8 weeks the polysiloxane coated polyetherurethane tubing, wet fatigued, exhibited greatly reduced surface cracking compared to the untreated control when viewed with SEM imaging.

At 8 weeks the polysiloxane coated polyetherurethane wet fatigued sample exhibited a smaller decrease in molecular weight compared to the uncoated polyetherurethane wet fatigued sample: 2.5 M₀/Mₙ vs. 8.7 M₀/Mₙ.

### Example 12—Polysiloxane (410 nm) Coated Polyetherurethane “Fatigued and Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 410+/−50 nanometers.

Polysiloxane coated polyetherurethane was “wet fatigued” using the Fatigue Test and then “aged” with the oxidation test. These samples were evaluated for appearance (optical and SEM imaging), and polyurethane molecular weight (GPC).

At 8 weeks the polysiloxane coated polyetherurethane tubing, wet fatigued, exhibited greatly reduced surface cracking compared to the untreated control when viewed with SEM imaging.

At 8 weeks the polysiloxane coated polyetherurethane wet fatigued sample exhibited a smaller decrease in molecular weight compared to the uncoated polyetherurethane wet fatigued sample: 2.3 M₀/Mₙ vs. 8.7 M₀/Mₙ.

### Example 13—Polysiloxane (265 nm) Coated Polyetherurethane “Fatigued and Aged”

PELLETHANE 80A polyetherurethane tubing was coated with a layer of polysiloxane with an HMDSO plasma in a batch reactor. The layer of polysiloxane covalently bonded with the polyetherurethane. The layer of polysiloxane had a uniform thickness of 265+/−50 nanometers.

Polysiloxane coated polyetherurethane was “wet fatigued” using the Fatigue Test and then “aged” with the oxidation test. These samples were evaluated for appearance (optical and SEM imaging), and polyurethane molecular weight (GPC).

At 8 weeks the polysiloxane coated polyetherurethane tubing, wet fatigued, exhibited greatly reduced surface cracking compared to the untreated control when viewed with SEM imaging.

At 8 weeks the polysiloxane coated polyetherurethane wet fatigued sample exhibited a smaller decrease in molecular weight compared to the uncoated polyetherurethane wet fatigued sample: 2.5 M₀/Mₙ vs. 8.7 M₀/Mₙ.

All references and publications cited herein are expressly incorporated herein by reference in their entirety into this disclosure, except to the extent they may directly contradict this disclosure. Illustrative embodiments of this disclosure are discussed, and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1. An implantable medical device comprising:
a contacting substrate for contacting a bodily fluid or tissue, the contacting substrate comprising polyetherurethane having a polyetherurethane surface, and
a layer of polysiloxane chemically bonded to the polyetherurethane surface.

2. The implantable medical device according to claim 1, wherein the contacting substrate is an elongated lead body, the elongated lead body comprising an electrical conductor, preferably the implantable medical device is a neurostimulation lead, a cardiac pacing lead, or a defibrillator lead.

3. The implantable medical device according to claim 2, wherein the contacting substrate has an outer polyetherurethane surface and an inner polyetherurethane surface, an inner polysiloxane layer is chemically bonded to the inner polyetherurethane surface, and an outer polysiloxane layer is chemically bonded to the outer polyetherurethane surface.

4. The implantable medical device according to claim 1, wherein the contacting substrate defines a tubular member, the tubular member is defined by an inner polyetherurethane cylindrical surface and an outer polyetherurethane cylindrical surface, the inner polyetherurethane cylindrical surface defining a cylindrical void space.

5. The implantable medical device according to claim 4, wherein an inner polysiloxane layer is chemically bonded to the inner polyetherurethane cylindrical surface, and an outer polysiloxane layer is chemically bonded to the outer polyetherurethane cylindrical surface.

6. The implantable medical device according to claim 1, wherein the polysiloxane layer is covalently bonded to the polyetherurethane surface.

7. The implantable medical device according to claim 1, wherein the layer of polysiloxane has a thickness of less than 900 nanometers.

8. The implantable medical device according to claim 1, wherein the polyetherurethane and layer of polysiloxane are extensible with an elastic elongation of at least 10%.

9. The implantable medical device according to claim 1, wherein the polysiloxane is a plasma polymerization reaction product of at least one monomer comprising hexamethyldisiloxane.

10. The implantable medical device according to claim 1, wherein the polyetherurethane has a Shore A hardness from 50 to 95.

11. A method of manufacturing an implantable medical device comprising:
plasma depositing a layer of polysiloxane onto a polyetherurethane surface, the layer of polysiloxane covalently bonded to the polyetherurethane surface forming a coated surface, and
assembling the coated surface into an implantable medical device.

12. The method according to claim 11, wherein the plasma depositing comprises plasma polymerizing the layer of polysiloxane onto a polyetherurethane surface.

13. The method according to claim 11, wherein the coated surface forms at least a portion of a lead body comprising an electrical conductor and assembling the lead body into an implantable medical device, preferably the implantable medical device is a neurostimulation lead, a cardiac pacing lead, or a defibrillator lead.

14. The method according to claim 11, wherein the polyetherurethane surface defines a tubular member, the tubular member comprising an inner polyetherurethane cylindrical surface and an outer polyetherurethane cylindrical surface, and the plasma depositing comprises plasma depositing an inner polysiloxane layer covalently bonded to the inner polyetherurethane cylindrical surface, and an outer polysiloxane cylindrical layer covalently bonded to the outer polyetherurethane cylindrical surface.

15. The method according to claim 11, wherein the plasma depositing comprises plasma depositing a layer of polysiloxane having a thickness of less than 750 nanometers.

16. The implantable medical device according to claim 3, wherein the inner polysiloxane layer separates the electrical conductor from the inner polyetherurethane surface.

17. The implantable medical device according to claim 4, wherein the tubular member forms an element of a catheter.

18. The implantable medical device according to claim 10, wherein the polyetherurethane has a Shore A hardness from 70 to 90, and the polyetherurethane has a polyether content in a range from about 40% to about 80%.

19. The method according to claim 14, wherein the implantable medical device comprises a catheter or a drug delivery device.

20. The implantable medical device according to claim 1, wherein the layer of polysiloxane has a thickness in a range from 200 nanometers to 750 nanometers, and the polyetherurethane, and layer of polysiloxane are extensible with an elastic elongation of at least 20%.
